SEQUENCE LISTING (2 sequences)

SEQ ID NO: 1 - DNA, 1428 bp, synthetic construct
  source 1..1428: mol_type = other DNA
tggttcctaaaaggttaccccaccgactttgggtgttacaaactctcatggtgtgacgggcggtgtgtacaaggcccgggaacgtattcaccgcggcatgctgatccgcgattactagcgattccgacttcatgtaggcgagttgcagcctacaatccgaactgagaatggctttaagagattagcttactctcgcgagttcgcaactcgttgtaccatccattgtagcacgtgtgtagcccaggtcataaggggcatgatgatttgacgtcatccccaccttcctccggtttgtcaccggcagtctcaccagagtgcccaacttaatgctggcaactgataataagggttgcgctcgttgcgggacttaacccaacatctcacgacacgagctgacgacaaccatgcaccacctgtatccatgtccccgaagggaacgtctaatctcttagatttgcatagtatgtcaagacctggtaaggttcttcgcgtagcttcgaattaaaccacatgctccaccgcttgtgcgggcccccgtcaattcctttgagtttcagccttgcggccgtactccccaggcggaatgcttaatgcgttagctgcagcactgaagggcggaaaccctccaacacttagcattcatcgtttacggtatggactaccagggtatctaatcctgtttgctacccatactttcgagcctcagcgtcagttacagaccagacagccgccttcgccactggtgttcttccatatatctacgcatttcaccgctacacatggagttccactgtcctcttctgcactcaagtttcccagtttccgatgcacttcttcggttgagccgaaggctttcacatcagacttaaaaaaccgcctgcgctcgctttacgcccaataaatccggacaacgcttgccacctacgtattaccgcggctgctggcacgtagttagccgtggctttctggttaaataccgtcaatacctgaacagttactctcagatatgttcttctttaacaacagagttttacgagccgaaacccttcttcactcacgcggcgttgctccatcagactttcgtccattgtggaagattccctactgctgcctcccgtaggagtttgggccgtgtctcagtcccaatgtggccgattaccctctcaggtcggctacgtatcattgccatggtgagccgttaccccaccatctagctaatacgccgcgggaccatccaaaagtgatagccgaagccatctttcaagctcggaccatgcggtccaagttgttatgcggtattagcatctgtttccaggtgttatcccccgcttctgggcaggtttcccacgtgttactcaccagttcgccactcacgcaaatgtaaatcatgatgcaagcaccaatcaataccagagttcgttcgactgc

SEQ ID NO: 2 - DNA, 1224 bp, synthetic construct
  source 1..1224: mol_type = other DNA
gatccgcgattactagcgattccgacttcatgtaggcgagttgcagcctacaatccgaactgagaatggctttaagagattagcttactctcgcgagttcgcaactcgttgtaccatccattgtagcacgtgtgtagcccaggtcataaggggcatgatgatttgacgtcatccccaccttcctccggtttgtcaccggcagtctcaccagagtgcccaacttaatgctggcaactgataataagggttgcgctcgttgcgggacttaacccaacatctcacgacacgagctgacgacaaccatgcaccacctgtatccatgtccccgaagggaacgtctaatctcttagatttgcatagtatgtcaagacctggtaaggttcttcgcgtagcttcgaattaaaccacatgctccaccgcttgtgcgggcccccgtcaattcctttgagtttcagccttgcggccgtactccccaggcggaatgcttaatgcgttagctgcagcactgaagggcggaaaccctccaacacttagcattcatcgtttacggtatggactaccagggtatctaatcctgtttgctacccatactttcgagcctcagcgtcagttacagaccagacagccgccttcgccactggtgttcttccatatatctacgcatttcaccgctacacatggagttccactgtcctcttctgcactcaagtttcccagtttccgatgcacttcttcggttgagccgaaggctttcacatcagacttaaaaaaccgcctgcgctcgctttacgcccaataaatccggacaacgcttgccacctacgtattaccgcggctgctggcacgtagttagccgtggctttctggttaaataccgtcaatacctgaacagttactctcagatatgttcttctttaacaacagagttttacgagccgaaacccttcttcactcacgcggcgttgctccatcagactttcgtccattgtggaagattccctactgctgcctcccgtaggagtttgggccgtgtctcagtcccaatgtggccgattaccctctcaggtcggctacgtatcattgccatggtgagccgttaccccaccatctagctaatacgccgcgggaccatccaaaagtgatagccgaagccatctttcaaactcggaccatgcggtccaagttgttatgcggtattagcatctgtttccaggtgttatcccc

(19) United States
(12) Patent Application Publication
(10) Pub. No.: US 2025/0034514 A1
(43) Pub. Date: Jan. 30, 2025

(54) LACTIPLANTIBACILLUS PLANTARUM EL6 FOR INCREASING FLAVONOID CONTENT, AND USE THEREOF

(71) Applicants: CHINA AGRICULTURAL UNIVERSITY, Beijing (CN); GUIZHOU UNIVERSITY, Guiyang (CN)

(72) Inventors: Kuikui NI, Beijing (CN); Yu GAO, Beijing (CN); Fuyu YANG, Beijing (CN)

(73) Assignees: CHINA AGRICULTURAL UNIVERSITY, Beijing (CN); GUIZHOU UNIVERSITY, Guiyang (CN)

(21) Appl. No.: 18/665,356

(22) Filed: May 15, 2024

(30) Foreign Application Priority Data
Jul. 24, 2023 (CN) 202310910779.X

(51) Int. Cl. C12N 1/20; A23K 10/18; A23K 10/30; C12R 1/25

(52) U.S. Cl. CPC C12N 1/205; A23K 10/18; A23K 10/30; C12R 2001/25

(57) ABSTRACT

The present disclosure relates to Lactiplantibacillus plantarum EL6 for increasing the flavonoids content, and a use thereof, and belongs to the technical field of microbial feeds. The present disclosure is intended to solve the problem that there is no special lactobacillus agent for increasing the flavonoids content during silage fermentation in the prior art. The Lactiplantibacillus plantarum EL6 in the present disclosure is a Gram-positive bacterium, which allows glucose facultative fermentation and has advantages such as acid resistance and high growth rate. In addition, the Lactiplantibacillus plantarum EL6 has an ability to allow the biotransformation of flavonoids, and can significantly increase a content of flavonoids in Medicago silage and promote the release and transformation of bound flavonoids. The Lactiplantibacillus plantarum EL6 can improve a fermentation quality of silage, and has advantages such as low cost, safety, reliability, and easy utilization.

### CROSS-REFERENCE TO RELATED APPLICATIONS

This application claims priority to Chinese Patent Application No. 202310910779.X filed on Jul. 24, 2023, the disclosure of which is hereby incorporated by reference in their entirety.

### SEQUENCE LISTING

The instant application contains a Sequence Listing which has been submitted electronically in XML format and is hereby incorporated by reference in its entirety. Said XML copy, created on May 6, 2024, is named LACTIPLANTIBACILLUS PLANTARUM EL6 FOR INCREASING FLAVONOID CONTENT, AND USE THEREOF.xml and is 5,176 bytes in size. The same sequence was previously submitted as part of priority application for instant application. The priority application, along with its Sequence Listing, was incorporated by reference in its entirety in the original filing of the instant application. No new matter is hereby added.

### TECHNICAL FIELD

The present disclosure relates to the technical field of microbial feeds, and in particular to Lactiplantibacillus plantarum EL6 for increasing the flavonoids content, and a use thereof.

### BACKGROUND

Medicago sativa L. is a perennial herbaceous plant of the pea family. Medicago sativa L. has advantages such as high protein content and excellent palatability, and includes a variety of active substances. Therefore, Medicago sativa L. is a high-quality forage source for ruminants. As a simple and low-cost forage storage method, silage can well preserve a nutritional value of a forage and improve the palatability and digestibility of a forage.

Silage fermentation can promote the release of active substances such as flavonoids. Flavonoids are a class of plant secondary metabolites with antimicrobial and antioxidant activities, and are important for animal health. A degree of fermentation dominated by a lactobacillus is a key to silage, but there is no special lactobacillus agent for increasing the flavonoids content in silage.

### SUMMARY

In view of the above analysis, the present disclosure is intended to provide Lactiplantibacillus plantarum EL6 for increasing the flavonoids content, and a use thereof. The present disclosure is intended to solve the problem that there is no special lactobacillus agent for increasing the flavonoids content during silage fermentation in the prior art.

In a first aspect, the present disclosure provides Lactiplantibacillus plantarum EL6 with an accession number of CGMCC No. 27566.

Further, the Lactiplantibacillus plantarum EL6 includes 16S rDNA shown in SEQ ID NO: 1.

Further, the Lactiplantibacillus plantarum EL6 is obtained through isolation from Medicago and cultivation.

In a second aspect, the present disclosure provides a silage additive including the Lactiplantibacillus plantarum EL6 described above.

In a third aspect, the present disclosure provides silage including the Lactiplantibacillus plantarum EL6 described above.

Further, the silage further includes Medicago silage and preferably, the Medicago silage is Medicago sativa L. silage.

In a fourth aspect, the present disclosure provides a preparation method of silage, including: mixing a silage raw material with the Lactiplantibacillus plantarum EL6 described above, and allowing fermentation to obtain the silage.

Further, a mass ratio of the Lactiplantibacillus plantarum EL6 to the silage raw material is larger than or equal to 1.0×10⁶ CFU/g.

Further, the silage raw material is Medicago sativa L.

In a fifth aspect, the present disclosure provides a use of the Lactiplantibacillus plantarum EL6 described above in preparation of silage.

Compared with the prior art, the present disclosure can allow at least one of the following beneficial effects:
(1) The Lactiplantibacillus plantarum EL6 in the present disclosure is a Gram-positive bacterium, which allows glucose facultative fermentation and has advantages such as acid resistance and high growth rate. In addition, the Lactiplantibacillus plantarum EL6 has an ability to allow the biotransformation of flavonoids, and can significantly increase a content of flavonoids in Medicago silage and promote the release and transformation of bound flavonoids.
(2) Compared with other conventional lactobacilli, the Lactiplantibacillus plantarum EL6 in the present disclosure can significantly improve a conversion efficiency of flavonoids during Medicago silage, increase the types and content of flavonoids in Medicago after silage, and directionally provide functional Medicago silage with a high quality and rich flavonoids.
(3) The Lactiplantibacillus plantarum EL6 of the present disclosure can improve a fermentation quality of silage, and has advantages such as low cost, safety, reliability, and easy utilization.

The above technical solutions in the present disclosure can also be combined with each other to provide increased preferred combination solutions. Other features and advantages of the present disclosure will be described in the following description, and some of these will become apparent from the description or be understood by implementing the present disclosure.

### DETAILED DESCRIPTION OF THE EMBODIMENTS

In a specific embodiment, the present disclosure discloses Lactiplantibacillus plantarum EL6, which was deposited in the China General Microbiological Culture Collection Center (CGMCC, Institute of Microbiology Chinese Academy of Sciences NO.1 West Beichen Road, Chaoyang District, Beijing, China) on Jun. 6, 2023, with an accession number of CGMCC No. 27566.

In an embodiment, the Lactiplantibacillus plantarum EL6 includes 16S rDNA shown in SEQ ID NO: 1.

In an embodiment, the Lactiplantibacillus plantarum EL6 is obtained through isolation from Medicago and cultivation.

Specifically, a process of the isolation and cultivation of the Lactiplantibacillus plantarum EL6 includes:

fresh Medicago is sterilized and ground, then 8.5% sterile normal saline is added, and a resulting supernatant is collected, transferred to an MRS medium, and cultivated.

In an embodiment, a growth temperature of the Lactiplantibacillus plantarum EL6 is 15° C. to 45° C., and further, the growth temperature is 30° C. to 35° C., such as 15° C., 20° C., 25° C., 30° C., 35° C., 40° C., or 45° C.

In another specific embodiment, the present disclosure discloses a silage additive including the Lactiplantibacillus plantarum EL6 described above.

In another specific embodiment, the present disclosure discloses silage including the Lactiplantibacillus plantarum EL6 described above.

In an embodiment, the silage further includes Medicago silage, and preferably; the Medicago silage is Medicago sativa L. silage.

In another specific embodiment, the present disclosure discloses a preparation method of silage, including: a silage raw material is mixed with the Lactiplantibacillus plantarum EL6 described above, and fermentation is allowed to obtain the silage.

In an embodiment, the silage raw material is Medicago sativa L.

In a preferred embodiment, a specific preparation method of the Medicago sativa L. silage is as follows:
(1) a Medicago sativa L. raw material to be fermented is chopped and thoroughly mixed;
(2) the Lactiplantibacillus plantarum EL6 is added to the Medicago sativa L. raw material; and
(3) a resulting mixture is vacuum-sealed and stored.

In an embodiment, in the step (1), the Medicago sativa L. raw material is chopped to 2 cm to 3 cm.

In an embodiment, in the step (3), after the storage is conducted at 20° C. to 25° C. for 6 d, a pH of the mixture drops to 4.08 to 4.35.

In an embodiment, a mass ratio of the Lactiplantibacillus plantarum EL6 to the Medicago raw material is larger than or equal to 1.0×10⁶ CFU/g.

As a further solution, the mass ratio of the Lactiplantibacillus plantarum EL6 to the Medicago raw material is 1.0×10⁶ CFU/g to 2.0× 10⁶ CFU/g.

In another specific embodiment, the present disclosure discloses a use of the Lactiplantibacillus plantarum EL6 described above in preparation of silage.

It should be noted that, compared with other conventional lactobacilli, the Lactiplantibacillus plantarum EL6 in the present disclosure can improve a conversion efficiency of flavonoids during Medicago silage, increase the types and content of flavonoids in Medicago after silage, and directionally provide functional Medicago silage with a high quality and rich flavonoids.

The technical solutions of the present disclosure are explained below in conjunction with specific examples.

The Medicago in the present disclosure is taken from Medicago sativa L. at an early flowering stage of the Ewenki Experimental Station of Ecological Grass-Animal Husbandry of the Chinese Academy of Sciences (East Longitude: 116.33°, and North Latitude: 39.98°) in Hulunbuir of the Inner Mongolia Autonomous Region.

### Example 1

### Isolation and Screening of a Strain EL6

1. A fresh Medicago material was placed in a sterile sampling bag and brought back to a laboratory, and 5 g to 10 g of complete Medicago plants were weighed and subjected to surface disinfection in a sterile environment on a clean bench as follows: the complete Medicago plants were soaked in 70% ethanol for 90 s, 3.25% to 4% sodium hypochlorite for 120 s, and 70% ethanol for 30 s, then rinsed 3 times with sterile water, and wiped with sterile filter paper to remove moisture on a surface. Sterile water obtained after the last rinsing was coated as a control. A sterilized material was placed in a sterilized mortar and ground (a small amount of a sterilized quartz sand was added to facilitate the grinding, and calcium carbonate was added for buffering), then 9 mL of 8.5% sterile normal saline was added, a resulting mixture was thoroughly stirred and then allowed to stand for 3 min. and 1 mL of a resulting supernatant (which was denoted as a 10⁻¹ concentration gradient) was taken and 10-fold diluted serially to 10⁻³. 100 μL of a dilution at each gradient was taken and coated on a plate with an MRS solid medium, and the plate was placed in an anaerobic bag and then incubated in a 30° C. or 37° C. incubator.

The MRS solid medium included the following components: peptone (Proteose peptone NO. 3): 10.0 g; beef extract: 10.0 g; yeast extract: 5.0 g: glucose (Dextrose): 20.0 g: Tween (Polysorbate 80): 1 mL; ammonium citrate: 2.0 g; sodium acetate (NaAc): 5.0 g; magnesium sulfate (MgSO⁴·7H₂O): 0.1 g; manganese sulfate (MnSO₄·4H₂O): 0.05 g; dipotassium phosphate (K₂HPO₄): 2.0 g; distilled water (H₂O): 1.000 mL; and 15 g/L agar. The MRS solid medium was sterilized at 121° C. for 20 min. After bacterial colonies grew; single colonies were picked, separated through repeated plate streaking until single colonies are obtained, then the single colonies are inoculated with an inoculation needle to a slope of an MRS solid medium test tube, and stored in a 4° C. refrigerator.
2. Gram staining: A specified amount of water was picked to a center of a glass slide, then a small amount of the strain cultivated above was picked with an inoculation loop and thoroughly mixed with water droplets on the glass slide, and a resulting mixture was coated to form a thin bacterial film and then naturally dried. The glass slide was fixed upwards above a slight fire, initially stained with crystal violet for 1 min, and then rinsed with water thoroughly (which should be gentle to prevent a water flow from directly impacting bacterial blocks). An iodine solution was added dropwise to the glass slide to allow mordant-staining for 1 min to 2 min. and then the glass slide was decolorized with 95% ethanol and washed with water. The glass slide was counter-stained with safranin for 1 min to 2 min. then washed with water, naturally dried, and then observed under a 1.000-amplification oil immersion lens. Bacteria stained blue-purple were Gram-positive bacteria, and bacteria stained red were Gram-negative bacteria.

Gram staining and cell shape observation were conducted. According to OD_{600 nm} and pH values of bacteria in MRS within 24 h. an endophytic lactobacillus (the strain EL6) with a high growth rate and a strong acid-producing ability was screened out, and a growth curve and related physiological indexes were determined in detail.

Results show that the strain EL6 is a Gram-positive bacillus with facultative fermentation, a high growth rate, a strong acid-producing ability, and strong acid and salt resistance. Physiological and biochemical characteristics are shown in Table 1.

Specific test methods for the physiological and biochemical properties are as follows:

Catalase experiment: A 3% (volume fraction) hydrogen peroxide solution was pipetted with a pipette tip and added to a plate, a small amount of bacteria was picked with an inoculation loop and thoroughly mixed with the hydrogen peroxide solution, and 2 min to 3 min later, the plate was observed. If there were bubbles, it is positive, and if there are no bubbles, it is negative.

Determination and screening of acid productions and growth rates of lactobacilli: An isolated and purified strain was inoculated into 3 mL of an MRS liquid medium and cultivated overnight on a shaker at 30° C. and 250 rpm for about 14 h to 16 h. A resulting bacterial solution was inoculated at an inoculum size of 1% (V/V) into 3 mL of a new MRS liquid medium and cultivated on a shaker at 30° C. and 250 rpm, and at 0 h. 2 h. 4 h. 6 h. 8 h. 10 h. 12 h, 14 h. 16 h. 18 h. 20 h. 22 h. and 24 h after the inoculation, a pH value of the MRS liquid medium was measured and an absorbance value was measured at a wavelength of 600 nm. 3 replicates needed to be set for each strain at each time point, and test tubes for cultivating bacteria needed to be of a same specification.

Temperature resistance test: A strain was inoculated at an inoculum size of 1% (V/V) into a new MRS liquid medium, and cultivated for 2 d at constant temperatures of 4° C. 15° C. and 45° C.

pH resistance test: A strain was inoculated at an inoculum size of 1% (V/V) into MRS liquid media with pH values of 3.0, 3.5, 4.0, 4.5, and 9.0, respectively, and cultivated for 2 d at 30° C. (a pH was adjusted with 2.0 M NaOH or 1.0 M HCL).

Salt resistance: A lactobacillus was inoculated into MRS liquid media with NaCl contents of 3% and 6.5%, respectively, and then cultivated in a 30° C. incubator for 2 d, and then a growth status of the lactobacillus was observed.

### Comparative Example 1

### Isolation and Screening of a Strain L3

A fresh Medicago material was placed in a sterile sampling bag and brought back to a laboratory. 20 g of complete Medicago plants were weighed and added to 180 mL of sterile normal saline in a sterile environment on a clean bench, and then shaken for 2 h on a shaker at 180 r/min and room temperature to obtain a stock solution with a concentration of 100 g·L⁻¹, which was denoted as a 10⁻¹ gradient. Then 1 mL of the stock solution was taken and added to a test tube including 9 mL of sterilized distilled water, and the test tube was thoroughly shaken to obtain a solution with a final concentration of 10 g·L⁻¹, which was denoted as a 10⁻² gradient. It was diluted by the same method to a 10⁻⁴ gradient. 100 μL of a dilution at each gradient was coated on a plate with an MRS solid medium, and cultivated in a 30° C. or 37° C. incubator. A formula of the MRS medium was the same as in Example 1. After bacterial colonies grew, single colonies were picked and separated through repeated plate streaking to obtain single colonies L3, and the single colonies were then inoculated with an inoculation needle to a slope of an MRS solid medium test tube, and stored in a 4° C. refrigerator.

Test methods of physiological and biochemical characteristics of the strain L3 in this comparative example were the same as in Example 1. Test results of the physiological and biochemical characteristics of the strain L3 are shown in Table 1.

TABLE 1
Physiological and biochemical characteristics
of strains EL6 and L3

| Strain | EL6 | L3 |
|---|---|---|
| Source | alfalfa endophyte | alfalfa epiphyte |
| Shape | Bacillus | Coccus |
| Gram staining | + | + |
| Catalase | − | − |
| Fermentation type | Facultative | Homolactic |

Growth temperature

| | | |
|---|---|---|
| 4° C. | w | w |
| 15° C. | +++ | ++ |
| 45° C. | + | ++ |

Growth pH

| | | |
|---|---|---|
| 3.0 | w | w |
| 3.5 | ++ | ++ |
| 4.0 | ++ | ++ |
| 4.5 | +++ | +++ |
| 9.0 | w | w |

Salt resistance

| | | |
|---|---|---|
| 3% NaCl | +++ | +++ |
| 6.5% NaCl | ++ | ++ |
| OD_{24 h} | 1.7122 | 1.6567 |
| pH_{24 h} | 3.76 | 3.76 |

Notes:
w: OD₆₀₀ nm < 0.5; +: 0.5 ≤ OD₆₀₀ nm < 1; ++: 1 ≤ OD₆₀₀ nm < 1.5; +++: 1.5 ≤ OD ₆₀₀ nm; and −: OD₆₀₀ nm < 0.5.

The strain EL6 screened in Example 1 and the strain L3 screened in Comparative Example 1 were subjected to homology analysis with a 16S rDNA gene

Each strain was inoculated into 5 mL of an MRS medium and cultivated overnight at 35° C., a resulting bacterial solution was transferred to a 1.5 mL centrifuge tube and centrifuged at 10,000 rpm/min for 3 min to 5 min to collect a bacterial pellet, the bacterial pellet was washed twice with TE 0.1 (10 mmol/L Tris-HCL, 0.1 mmol/L EDTA, pH 8.0) and then subjected to DNA extraction with a TIANamp Bacteria DNA Kit (TIANGEN BIOTECH CO., LTD., Beijing, China), and the absorbance OD_{600 nm} was detected. Then, PCR amplification was conducted. Amplification primers for 16S rDNA were 27f and 1492r (Monis et al., 2005), and a PCR procedure was as follows: 95° C. (5 min), −94° C. (30 s), −55° C. (1 min), −72° C. (1.5 min), and −72° C. (10 min), where there were 30 cycles of 94° C. (30 s), −55° C. (1 min), and −72° C. (1.5 min).

Amplification products were sent to Majorbio (China) for sequencing. Sequencing results were subjected to alignment in the gene bank of NCBI to find out a standard Lactiplantibacillus plantarum strain with a close relationship to a corresponding strain. Through the DNAman software, a screened strain was subjected to similarity analysis with a standard strain in terms of a partial sequence (about 1,400 bp to 1,500 bp) of 16S rDNA. 16S rDNA of the strain EL6 was shown in SEQ ID NO: 1, and the similarity between the strain EL6 and Lactiplantibacillus plantarum was more than 99%. Thus, in combination with physiological and biochemical indexes, it was determined that the strain EL6 was of the same species as Lactiplantibacillus plantarum. The 16S rDNA of the strain L3 was shown in SEQ ID NO: 2, and the similarity between the strain L3 and Pediococcus pentosaceus was more than 99%. In combination with physiological and biochemical indexes, it was determined that the strain L3 was of the same species as Pediococcus pentosaceus.

The Lactiplantibacillus plantarum EL6 in the present disclosure is the strain EL6 isolated and screened in Example 1, and the Lactobacillus pentosus L3 is the strain L3 isolated and screened in Comparative Example 1.

### Use Example 1

Use Test of the Lactiplantibacillus plantarum EL6 in Silage

Medicago was cut to 2 cm to 3 cm and then thoroughly mixed. Nutritional components of the Medicago raw material are shown in Table 1. The Lactiplantibacillus plantarum EL6 obtained in Example 1 and the Lactobacillus pentosus L3 obtained in Comparative Example 1 each were inoculated into the Medicago raw material at an amount of about 1×10⁶ CFU/g. The Lactiplantibacillus plantarum EL6 was not added in a CK group. A resulting Medicago material in each treatment group was thoroughly mixed and then packed in four 28 cm×35 cm polyethylene silage bags with about 500 g of the Medicago material in each bag, the polyethylene silage bags were vacuumed by a vacuum sealing machine for sealing and then stored at room temperature of 20° C. to 25° C. to allow fermentation, and 7 d, 15 d, and 60 d after the fermentation, a bag was opened for sampling to analyze a fermentation quality and nutritional components of Medicago silage. Analysis results are shown in Tables 2 to 4. Changes in the flavonoids content after 60 d of silage with the strains EL6 and L3 are shown in Table 5.

In addition, a method for analyzing components in the Medicago silage and raw material are as follows:

20 g of a Medicago silage sample was taken. 180 mL of distilled water was added to obtain a mixture, the mixture was thoroughly stirred, then crushed with a tissue crusher for 1 min, and then filtered with four layers of gauze to obtain a leaching residue and a leaching liquor, and the leaching liquor was used for determination of pH and lactic acid (LA), acetic acid (AA), butyrate acid acid (BA), and ammonia nitrogen contents. A pH value of a Medicago silage leaching liquor was determined by a pH meter (pHS-3C. Shanghai, China). The LA. AA, and BA contents were analyzed by high-performance liquid chromatography under the following conditions: a chromatographic column: a KC-811 column, a detector: SPD-M10AVP, a detection wavelength: 210 nm, a mobile phase: 3 mmol/L perchloric acid, a flow rate: 1 mL/min. an injection volume: 5 μL. and a column temperature: 50° C. The ammonia nitrogen was determined by a phenol-sodium hypochlorite colorimetric method (Broderick and Kang. 1980). Dry matter (DM) contents in the Medicago silage raw material and silage were determined by an oven-drying method as follows: a sample was thoroughly mixed and then oven-dried in a 65° C. blast drying oven for about 48 h until a mass of the sample was constant, and a dry matter content was determined. An oven-dried sample was crushed by a plant crusher, then sieved, and then tested for crude protein (CP), ether extract (EE), neutral detergent fiber (NDF), and acid detergent fiber (ADF) contents. The CP content was determined by the Kjeldahl nitrogen determination method, the EE content was determined by the petroleum ether extraction method (AOAC, 2010), and the NDF and ADF contents were determined by the Van's detergent fiber method (Van Soest et al., 1991).

A preparation method of a silage sample for flavonoids-targeted metabolism analysis was as follows: a Medicago silage sample was vacuum-lyophilized and then milled with a ball mill (30 HZ. 1.5 min) to obtain a powder: 20 mg of the powder was weighed. 10 μL of an internal standard mixed working solution with a concentration of 4.000 nmol/L and 500 μL of 70% methanol were added to the weighed powder to obtain a mixture, and the mixture was ultrasonically-treated for 30 min and then centrifuged at 4° C. and 12.000 r/min for 5 min to obtain a supernatant; and the supernatant was collected, filtered through a 0.22 μm filter membrane, and then injected into a sample bottle for LC-MS/MS analysis. Chromatography-mass spectrometry acquisition conditions: high-performance liquid chromatography (ExionLC™ AD) and tandem mass spectrometry (QTRAP® 6500+). Liquid chromatography conditions: a chromatographic column: Waters ACQUITY UPLC HSS T3 C18 column (1.8 μm, 100 mm×2.1 mm i.d.): a mobile phase A: ultrapure water+0.05% formic acid, and a mobile phase B: acetonitrile+0.05% formic acid; a flow rate: 0.35 mL/min; a column temperature: 40° C.; an injection volume; 2 μL; and an elution gradient: 0 min: A/B: 90:10 (V/V), 1 min: A/B: 80:20 (V/V), 9 min: 30:70 (V/V), 12.5 min: A/B 5:95 (V/V), 13.5 min: 5:95 (V/V), 13.6 min: 90:10 (V/V), and 15 min: 90:10 (V/V). A Metware Database was constructed based on standards to allow the qualitative analysis of mass spectrometry data. A multi-reaction monitoring mode of triple quadrupole mass spectrometry was used to allow the quantitative analysis.

TABLE 2
Nutritional components of the Medicago raw material before silage

| | alfalfa |
|---|---|
| Item | raw material |
| DM (g/kg FM) | 502.43 |
| WSCs (g/kg DM) | 84.25 |
| EE (g/kg DM) | 12.72 |
| CP (g/kg DM) | 234.24 |
| NDF (g/kg DM) | 378.65 |
| ADF (g/kg DM) | 257.81 |

Notes:
FM: fresh matter, and DM: dry matter. WSC: water soluble carbohydrates, EE: ether extract, CP: crude protein, NDF: neutral detergent fiber, ADF: acid detergent fiber.

TABLE 3
Impact of the Lactiplantibacillus plantarum EL6 additive
on a fermentation quality of Medicago silage

| Item | T | | CK | EL6 |
|---|---|---|---|---|
| LA | 7 | d | 20.990 | 38.284 |
| (g/kg DM) | 15 | d | 22.985 | 42.822 |
| | 60 | d | 36.692 | 46.328 |
| AA | 7 | d | 16.613 | 27.637 |
| (g/kg DM) | 15 | d | 19.023 | 26.597 |
| | 60 | d | 23.688 | 27.773 |
| BA | 7 | d | 6.957 | 7.791 |
| (g/kg DM) | 15 | d | 9.816 | 11.631 |
| | 60 | d | 15.642 | 16.041 |
| pH | 7 | d | 4.527 | 4.353 |
| | 15 | d | 4.213 | 4.170 |
| | 60 | d | 4.123 | 4.083 |
| NH₃-N/TN | 7 | d | 0.057 | 0.074 |
| (g/kg DM) | 15 | d | 0.179 | 0.179 |
| | 60 | d | 0.227 | 0.247 |

Notes:
ND: lower than a detection level, T: treatment, DM: dry matter, LA: lactic acid, AA: acetic acid, BA: butyrate acid, NH₃-N/TN: Ammonia nitrogen/total nitrogen.

TABLE 4
Impact of the Lactiplantibacillus plantarum EL6 additive
on nutritional components of Medicago silage

| Item | T | | CK | EL6 |
|---|---|---|---|---|
| DM | 7 | d | 561.735 | 565.250 |
| (g/kg FM) | 15 | d | 543.721 | 559.800 |
| | 60 | d | 549.358 | 557.708 |
| WSC | 7 | d | 18.173 | 19.408 |
| (g/kg DM) | 15 | d | 13.809 | 21.400 |
| | 60 | d | 15.073 | 25.042 |
| EE | 7 | d | 21.920 | 22.889 |
| (g/kg DM) | 15 | d | 22.778 | 27.751 |
| | 60 | d | 29.180 | 27.154 |
| NDF | 7 | d | 359.861 | 350.969 |
| (g/kg DM) | 15 | d | 372.096 | 350.735 |
| | 60 | d | 370.407 | 361.675 |
| ADF | 7 | d | 270.134 | 262.309 |
| (g/kg DM) | 15 | d | 278.724 | 267.395 |
| | 60 | d | 281.632 | 269.459 |
| CP | 7 | d | 187.990 | 200.658 |
| (g/kg DM) | 15 | d | 197.680 | 200.128 |
| | 60 | d | 188.961 | 204.902 |

Notes:
FM: fresh matter, and DM: dry matter, WSC: water soluble carbohydrates, EE: ether extract, CP: crude protein, NDF: neutral detergent fiber, ADF: acid detergent fiber.

TABLE 5
Flavonoids contents significantly increased after 60 d of Medicago
silage with EL6 and L3 compared with natural Medicago silage

| Substance | CK | L3 | EL6 |
|---|---|---|---|
| Scutellarin | 0.3520 | 0.4472 | 0.4223 |
| Tectochrysin | 0.0054 | 0.0051 | 0.0065 |
| Acacetin | 0.0762 | 0.0718 | 0.1925 |
| Apigenin-7-glucuronic acid | 1.3085 | 1.4185 | 1.6207 |
| Eupatorin | 0.3458 | 0.2765 | 0.4277 |
| 3,4′-Dihydroxyflavone | 0.0103 | 0.0055 | 0.0125 |
| Genkwanin | 0.0948 | 0.0939 | 0.1131 |
| 4,4′-Dimethoxychalcone | 0.0021 | 0.0041 | 0.0044 |
| Phloretin | 0.0113 | 0.0098 | 0.0127 |
| Pinocembrin | 0.3136 | 0.3055 | 0.3401 |
| Farrerol | 0.0203 | 0.0354 | 0.0469 |
| Dihydromyricetin | 0.1666 | 0.1605 | 0.1979 |
| Aromadendrin | 3.3572 | 3.3941 | 3.5380 |
| Quercitrin | 0.0498 | 0.1833 | 0.1409 |
| Kaempferol | 34.9054 | 32.473 | 43.0724 |
| 5-Deoxykaempferol | 0.0000 | 4.1831 | 5.6642 |
| Isorhamnetin | 3.3240 | 2.7455 | 3.7798 |
| (−)-Gallocatechin | 0.0794 | 0.0971 | 0.0955 |
| Tectorigenin | 1.4587 | 1.4732 | 1.6743 |
| Prunetin | 3.3765 | 3.3869 | 3.9570 |
| Genistin | 0.2968 | 0.3436 | 0.3735 |

It can be seen from Tables 2 to 5 that, compared with the control group, the Lactiplantibacillus plantarum EL6 additive can increase the contents of LA, AA, and BA in Medicago silage and reduce the pH value of Medicago silage, and can also well preserve DMs and crude proteins. Compared with the silage raw material, the addition of the Lactiplantibacillus plantarum EL6 can significantly increase the contents of 21 kinds of flavonoids, and compared with the addition of Lactobacillus pentosus L3, the addition of the Lactiplantibacillus plantarum EL6 can significantly increase the contents of 18 kinds of flavonoids. Therefore, the Lactiplantibacillus plantarum EL6 is a novel functional additive that can both improve a quality of Medicago silage and efficiently transform flavonoids, and has characteristics such as multiple effects, strong effects, and low cost.

The above are merely preferred specific implementations of the present disclosure, but the protection scope of the present disclosure is not limited thereto. Any modification or replacement easily conceived by those skilled in the art within the technical scope of the present disclosure should fall within the protection scope of the present disclosure.

1. Lactiplantibacillus plantarum EL6 with an accession number of CGMCC No. 27566, wherein the Lactiplantibacillus plantarum EL6 is obtained through isolation from Medicago and cultivation.

2. The Lactiplantibacillus plantarum EL6 according to claim 1, wherein the Lactiplantibacillus plantarum EL6 comprises 16S rDNA shown in SEQ ID NO: 1.

3. Silage comprising the Lactiplantibacillus plantarum EL6 with an accession number of CGMCC No. 27566.

4. The silage according to claim 3, further comprising Medicago silage, wherein preferably, the Medicago silage is Medicago sativa L. silage.

5. A preparation method of silage, comprising: mixing a silage raw material with the Lactiplantibacillus plantarum EL6 with an accession number of CGMCC No. 27566, and allowing fermentation to obtain the silage.

6. The preparation method of silage according to claim 5, wherein a mass ratio of the Lactiplantibacillus plantarum EL6 to the silage raw material is larger than or equal to 1.0×106 CFU/g.

7. The preparation method of silage according to claim 5, wherein the silage raw material is Medicago sativa L.
